(19) United States
(12) Patent Application Publication
(10) Pub. No.: US 2022/0280425 A1
(43) Pub. Date: Sep. 8, 2022

(54) FLAVONOIDS AND LINOLEIC ACID USES IN HYPERTENSION

(71) Applicant: King Abdulaziz University, Jeddah (SA)

(72) Inventors: Hossam M. Abdallah, Jeddah (SA); Hany Mohamed El-Bassossy, Zagazig (EG); Ali M. El-Halawany, Cairo (EG); Tarek A. Ahmed, Jeddah (SA); Gamal A. Mohamed, Jeddah (SA); Azizah M. Malebari, Jeddah (SA); Noura Ahmed Hassan, Zagazig (EG)

(21) Appl. No.: 17/190,504

(22) Filed: Mar. 3, 2021

(51) Int. Cl. A61K 9/107; A61K 47/12; A61K 47/10; A61K 47/26; A61P 9/08; A61K 31/353; A61P 9/12

(52) U.S. Cl. CPC A61K 9/1075; A61K 47/12; A61K 47/10; A61K 47/26; A61P 9/08; A61K 31/353; A61P 9/12; A61B 5/021

(57) ABSTRACT

Methoxylated flavonoids are described for their compositions and uses in hypertension treatment as vasodilators. The flavonoids are intravenously administered in a self-nanoemulsifying drug delivery system (SNEDDS) in combination with linoleic acid which provides lowering heart rate, thus suppressing reflex tachycardia elicited by the vasodilating effect of flavonoids. The compositions are used to treat or prevent acute hypertension or any acute symptoms of emergency hypertension.

### FIELD OF THE INVENTION

The invention relates to pharmaceutical compositions of flavonoids in a linoleic acid containing self-nanoemulsifying drug delivery system (SNEDDS) and methods of using such compositions as therapeutic agents for hypertension while suppressing reflex tachycardia.

### BACKGROUND OF THE INVENTION

Hypertension is one of the most important risk factors of cardiovascular disorder and the biggest contributor to the global burden of disease. In early stages of hypertension, changes in lifestyle (e.g. weight loss, physical exercise, decreased salt intake, reducing alcohol intake, healthy diet, etc.) are shown to be effective in lowering blood pressure and decrease the risk of developing severe form of hypertension. When the lifestyle and behavioral changes are not sufficient, anti-hypertensive pharmacological treatments are prescribed to decrease the likelihood of cardiovascular events such as heart attack, heart failure and stroke, which may be further associated as symptoms of hypertensive emergency.

Hypertensive emergency is defined as an elevation of systolic blood pressure over 180 mmHg and/or diastolic blood pressure exceeding 120 mm Hg with the presence of acute target organ damage (e.g. kidney insult, encephalopathy, retinal hemorrhage, etc.). Although hypertensive emergency is rare, it is often developed when hypertension goes untreated, the patient does not take their blood pressure medication, was unaware of the condition, or caused by other blood pressure increasing substances. One of the treatments for hypertensive emergencies includes vasodilators, which are effective and widely used to reduce symptoms of systemic hypertension, such as myocardial infarction, angina, heart failure, stroke, chronic kidney disease and preeclampsia. Several different classes of vasodilators exist (e.g. ACE inhibitors, angiotensin receptor blockers, nitrates, calcium channel blockers, minoxidil and hydralazine); but some of these drugs bring severe adverse effects (e.g. fatigue, cardiac dysfunction, skin rash, sexual dysfunction, depression, etc.). One of the adverse effects of vasodilator is reflex tachycardia, in which the heart rate exceeds the normal resting rate in response to an effective vasodilator-induced lower heart rate by a feedback mechanism of the body trying to maintain adequate blood flow and blood pressure. Thus, vasodilators are often used in conjunction with a beta-blocker or diuretic to attenuate the baroreceptor-mediated reflex tachycardia and renal sodium retention, respectively.

In recent years, the use of bioactive compounds from plant sources has been actively researched, owing to their efficacy, safety, relative availability and low cost. Almost two hundred molecules, including flavonoids, alkaloids and terpenoids, have been identified as bioactive compounds from plants. In particular, several biologically active compounds (e.g. diterpenes, flavonoids, phenylpropanoids, coumarins, terpenoids, etc.) were extracted from a small shrub called Psiadia punctulata (PP), which is usually found in Eritrea, Saudi, Arabia and North East India, for their uses in asthma, abdominal pains, analgesic effects, colds, malaria, phrenic neuromuscular nerve blocking and smooth muscle relaxation. To this end, methanol extract of the whole PP plant have also been reported to show a blood pressure lowering effect, which may suggest potential function as a vasodilator. However, the effects and functions of such flavonoids in hypertensive conditions, especially in a hypertensive emergency, are unknown.

Further, the pharmacokinetics of many flavonoids have severely limited in their therapeutic usefulness and a vast number of flavonoids, especially synthetic flavonoids, tend to be highly lipid soluble molecules, thus leading to a difficulty in administration as therapeutic agents. For these lipophilic molecules, selection of lipid carriers (e.g. SNEDDS) based on their effects in composition size and effect are required.

There is a need in the art for a novel vasodilator in an improved delivery system. The ideal vasodilator with appropriate pharmacokinetics that is more potent and more targeted yet without the adverse effect (e.g. reflex tachycardia) is desired. Further, more natural form of therapeutics with proven safety and relative availability are needed to further reduce the unwanted side effects of synthetic therapeutics.

### SUMMARY OF THE INVENTION

The disclosure provides pharmaceutical compositions of flavonoids in a self-nanoemulsifying drug delivery system (SNEDDS) and methods of using such system for treating hypertension. In particular, the invention provides linoleic acid as a component of SNEDDS and the use of flavonoids in a linoleic acid containing SNEDDS for treating hypertension while suppressing reflex tachycardia. The bioactive flavonoid compounds described herein are advantageously safe, less expensive, and more readily available than synthetic compounds or other currently available vasodilators.

In an aspect of the invention, a composition of a methoxylated flavonoid, or a pharmaceutically acceptable salt or solvate thereof, in a linoleic acid based SNEDDS in which the flavonoid is represented by a general formula I, is provided:

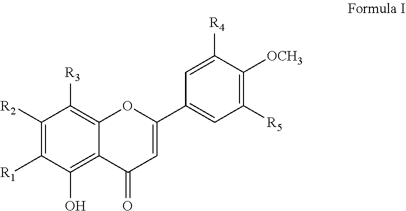

wherein, R₁, R₂, R₃, R₄, and R₅ are each independently selected from the group consisting of: alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone; or two adjacent substituents selected from R₁, R₂, R₃, R₄, and R₅ are connected in a 5 or 6-membered ring to form a bicyclic aryl or bicyclic heteroaryl.

In some aspects of the invention, the methoxy flavonoid in a linoleic acid based SNEDDS is umuhengerin, which is represented by formula Ia:

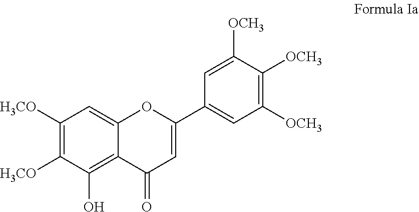 [SMILES: COc1cc(-c2cc(=O)c3c(O)c(OC)c(OC)cc3o2)cc(OC)c1OC]

In other aspects of the invention, the methoxy flavonoid in a composition is 5,3′-dihydroxy-6,7,4′,5′-tetramethoxyflavone and is represented by formula Ib:

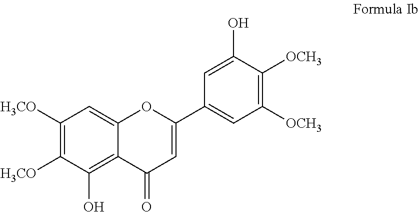 [SMILES: COc1cc(-c2cc(=O)c3c(O)c(OC)c(OC)cc3o2)cc(O)c1OC]

In these embodiments, SNEDDS comprises 70-90% of PEG 400 and 5-20% of 80% tween. In preferred embodiments, SNEDDS comprises 5-30% of linoleic acid or functionally similar component thereof (e.g. olive oil, linoleic acid, oleic acid, etc.).

Embodiments of the invention also contemplate use of such compositions for preventing or combating hypertension as well as in other clinical applications. In some embodiments, the present invention provides a method of preventing and/or treating hypertension in a subject in need thereof, especially in which the subject is at risk of developing reflex tachycardia in response to a vasodilator treatment. The method comprises a step of administering an effective amount of at least one flavonoid specified above (i.e. formula I, formula Ia and/or formula Ib) in a linoleic acid containing SNEDDS. Methods of invention also include administering to a mammal (e.g., a human) in need thereof a therapeutically effective amount of linoleic acid as a component of SNEDDS. In these embodiments, linoleic acid is used for a particular function in reduction of heart rate, leading to prevention of a reflex tachycardia associated with vasodilators, in this case, flavonoids.

When used to treat symptoms of hypertension in a human or an animal subject, the subject is administered with one or multiple doses of said compositions, in which the composition may be administered via any suitable route, but preferably, via intravenous injection. In another aspect, the composition is delivered to a subject, and the subject in need of such treatment is suffering acute hypertension. In another aspect of the invention, the acute condition is selected from stroke, myocardial infarction, mechanical trauma resulting from crush injury, heart bypass, heart transplant or vascular surgery.

Additional features and advantages of the present invention will be set forth in the description of disclosure that follows, and in part will be apparent from the description of may be learned by practice of the disclosure. The disclosure will be realized and attained by the compositions and methods particularly pointed out in the written description and claims hereof.

### BRIEF DESCRIPTION OF THE DRAWINGS

FIG. 1 Isolated compounds (1-5) from Psiadia punctulata

FIG. 2A Representative traces of blood hemodynamic invasive recording.

FIG. 2B Representative traces of blood hemodynamic invasive recording before slow intravenous injection.

FIG. 2C Representative traces of blood hemodynamic invasive recording before slow intravenous injection of formulation 1 in dose of 12 ug/kg in angiotensin model of hypertensive rats.

FIG. 2D Representative traces of blood hemodynamic invasive recording before slow intravenous injection of formulation 1 in dose of 24 ug/kg in angiotensin model of hypertensive rats.

FIG. 2E Representative traces of blood hemodynamic invasive recording before slow intravenous injection of formulation 1 in dose of 36 ug/kg in angiotensin model of hypertensive rats.

FIG. 3A Effect on slow intravenous injection of formulations 1 and 5 on the systolic blood pressure (SBP). Data presented as mean ±standard error of 6 animals. *P<0.05, compared with the corresponding vehicle control values.

FIG. 3B Effect on slow intravenous injection of formulations 1 and 5 on the diastolic blood pressure (DBP). Data presented as mean ±standard error of 6 animals. *P<0.05, compared with the corresponding vehicle control values.

FIG. 3C Effect on slow intravenous injection of formulations 1 and 5 on the heart rate. Data presented as mean ±standard error of 6 animals. *P<0.05, compared with the corresponding vehicle control values.

FIG. 4A Effect on slow intravenous injection of formulations 1 and 5 on the pulse pressure. Data presented as mean ±standard error of 6 animals. *P<0.05, compared with the corresponding vehicle control values.

FIG. 4B Effect on slow intravenous injection of formulations 1 and 5 on dicrotic notch pressure. Data presented as mean ±standard error of 6 animals. *P<0.05, compared with the corresponding vehicle control values.

FIG. 4C Effect on slow intravenous injection of formulations 1 and 5 on the systolic blood pressure-dicrotic notch pressure difference (SDP-difference). Data presented as mean ±standard error of 6 animals. *P<0.05, compared with the corresponding vehicle control values.

FIG. 5A Effect on slow intravenous injection of formulations 1 and 5 on the ejection duration. Data presented as mean ±standard error of 6 animals. *P<0.05, compared with the corresponding vehicle control values.

FIG. 5B Effect on slow intravenous injection of formulations 1 and 5 on non-ejection duration. Data presented as mean ±standard error of 6 animals. *P<0.05, compared with the corresponding vehicle control values.

FIG. 5C Effect on slow intravenous injection of formulations 1 and 5 on peak time. Data presented as mean ±standard error of 6 animals. *P<0.05, compared with the corresponding vehicle control values.

FIG. 6A Effect on slow intravenous injection of formulations 1 and 5 on P wave duration. Data presented as mean ±standard error of 6 animals. *P<0.05, compared with the corresponding vehicle control values.

FIG. 6B Effect on slow intravenous injection of formulations 1 and 5 on PR interval. Data presented as mean ±standard error of 6 animals. *P<0.05, compared with the corresponding vehicle control values.

FIG. 7A Effect on slow intravenous injection of formulations 1 and 5 on the OTc interval. Data presented as mean ±standard error of 6 animals. *P<0.05, compared with the corresponding vehicle control values.

FIG. 7B Effect on slow intravenous injection of formulations 1 and 5 on the JT interval. Data presented as mean ±standard error of 6 animals. *P<0.05, compared with the corresponding vehicle control values.

FIG. 8A Effect on slow intravenous injection of formulations 1 and 5 on the R amplitude. Data presented as mean ±standard error of 6 animals. *P<0.05, compared with the corresponding vehicle control values.

FIG. 8B Effect on slow intravenous injection of formulations 1 and 5 on the T amplitude (B). Data presented as mean ±standard error of 6 animals. *P<0.05, compared with the corresponding vehicle control values.

### DETAILED DESCRIPTION

The preferred embodiments of the present disclosure are directed toward compositions of methoxy flavonoids and linoleic acid in a self-nanoemulsifying drug delivery system (SNEDDS) and methods of using such compositions as therapeutic treatments for hypertensive emergencies without triggering reflex tachycardia.

As used herein, the term “self-nanoemulsifying drug delivery system” means anhydrous homogenous liquid mixtures consisting of oil, surfactant, drug and co-emulsifier or solubilizer which spontaneously form oil-in-water nanoemulsion of approximately 20-200 nm (or less than 20 nm) in size upon dilution with water under gentle agitation. The composition of the SNEDDS may be optimized with the help of phase diagrams and to improve bioavailability of hydrophobic drugs by several routes of administration. In addition, the SNEDDS formulations may be filled in soft and hard gelatin capsules. In some embodiments, SNEDDS comprises 70-90% of PEG 400 and 5-20% of 80% tween. In another embodiments, SNEDDS further comprises 5-30% of linoleic acid or the like functional component (e.g. olive oil, linoleic acid, oleic acid, etc.). The SNEDDS described herein may be used in any of a variety of therapeutic applications, e.g. 1) they may be administered through numerous routes, examples of routes include but are not limited to intranasal, oral-route forms such as gel capsules granules or suspensions or solutions, sublingual and buccal administration forms, aerosols, implants, subcutaneous, transdermal, topical, intraperitoneal, intramuscular, intravenous, subdermal, transdermal, intrathecal, intraduodenal, and rectal administration forms, 2) they may be used as a treatment of CNS-related disorders such as acute stroke, cerebral vascular ischemia, cerebral dysfunction and Alzheimer's disease and/or used as a supplement for improving symptoms of dementia, energy metabolism, weight loss, and anti-aging.

As used herein, the term “flavonoids” refers to a class of polyphenolic secondary metabolites found in plants. The flavonoids generally comprise a structure of a 15-carbon skeleton and further comprises of two phenyl rings and a heterocyclic ring. Flavonoids may be further classified into bioflavonoids, isoflavonoids and neoflavonoids. The aqueous solubility of flavonoids, especially methoxy flavonoids, is low. Thus, the methoxy flavonoids are preferably formulated for intravenous administration.

In one embodiment, the invention provides a composition of a methoxylated flavonoid in a linoleic acid based SNEDDS, in which the flavonoid is represented by a general formula I:

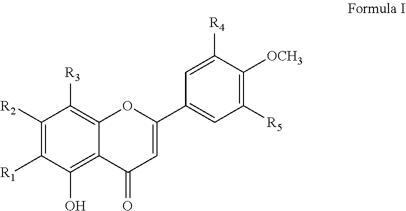

wherein, R₁, R₂, R₃, R₄, and R₅ are each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone; or two adjacent substituents selected from R₁, R₂, R₃, R₄, and R₅ are connected in a 5 or 6-membered ring to form a bicyclic aryl or bicyclic heteroaryl.

In this embodiment, the methoxy flavonoids were extracted in methanol from dried aerial parts of P. punctulate. Total methanol extract was suspended in the last amount of water and extracted with chloroform. The chloroform fraction was chromatographed on silica gel column or RP₁₈ column and eluted with various running solvents (e.g. hexane, EtOAc, MeOH).

In preferred embodiments, a composition of umuhengerin in a linoleic acid based SNEDDS is represented by formula Ia:

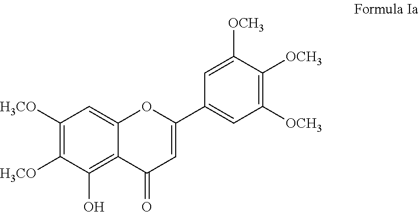 [SMILES: COc1cc(-c2cc(=O)c3c(O)c(OC)c(OC)cc3o2)cc(OC)c1OC]

In another preferred embodiments, a composition of 5,3′-dihydroxy-6,7,4′,5′-tetramethoxyflavone in a linoleic acid based SNEDDS is represented by formula Ib:

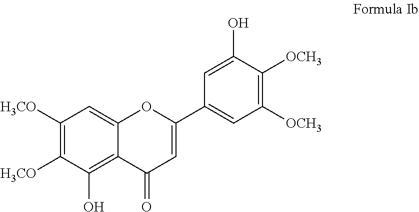 [SMILES: COc1cc(-c2cc(=O)c3c(O)c(OC)c(OC)cc3o2)cc(O)c1OC]

Certain derivatives or chemically modified versions of methoxy flavonoid compounds are also encompassed and can be used in the practice of the invention, examples of which include but are not limited to: the replacement of OH groups by e.g. SH; the modification of OH or OCH₃ groups, e.g. by esterification, acetylation, amidation, bonding to protective groups such as Acetyl, Benzoyl, Benzyl, β-Methoxyethoxymethyl ether (MEM), Dimethoxytrityl, [bis-(4-methoxyphenyl)phenylmethyl] (DMT), Methoxymethyl ether (MOM), Methoxytrityl [(4-methoxyphenyl)diphenylmethyl] (MMT), p-Methoxybenzyl ether (PMB), p-Methoxyphenyl ether (PMP), Methylthiomethyl ether, Pivaloyl (Piv), Tetrahydropyranyl (THP), Tetrahydrofuran (THF), Trityl (triphenylmethyl, Tr), Silyl ether (including trimethylsilyl (TMS), tert-butyldimethylsilyl (TBDMS), tri-iso-propylsilyloxymethyl (TOM), and triisopropylsilyl (TIPS) ethers), Methyl ethers and Ethoxyethyl ethers (EE), e.g. to create a prodrug form and/or to slow, enhance or target absorption. COOH groups can also be modified.

In some aspects of the invention, 1-50% of linoleic acid, or more preferably 3-30% of linoleic acid, and most preferably 5-20% of linoleic acid is incorporated into a SNEDDS to manufacture methoxy flavonoid compositions. In these embodiments, the linoleic acid provides antiarrhythmic function to regulate the heart rate. When administered together with the methoxy flavonoids, the linoleic acid suppresses reflex tachycardia stimulated by vasodilating effect of flavonoids, thus greatly enhance the efficacy of the composition as a hypertensive treatment.

In the most preferred embodiment, one or more methoxy flavonoids and linoleic acid is in SNEDDS. In another embodiment, one or more methoxy flavonoids are free of linoleic acid in a composition. In yet another embodiment, linoleic acid and non-flavonoid vasodilator are in a SNEDDS. In some embodiments, flavonoid compounds and linoleic acid may be incorporated into another pharmaceutically acceptable carrier in a form suitable for therapeutic delivery to a subject. Another carrier or suspension for injections includes sterile saline, bacteriostatic water, Cremophor EL (BASF, Parsippany, N.J.), phosphate buffered saline (PBS), ethanol or polyol (for example, glycerol, propylene glycol and polyethylene glycol and the like). The term “pharmaceutically acceptable carrier” as used herein refers to any and all solvents, dispersion media, coatings, isotonic and absorption delaying agents, and the like, that are compatible with pharmaceutical administration. The compositions may also contain other active compounds providing supplemental, additional, or enhanced therapeutic functions.

In some aspects of the invention, one or more flavonoid derivatives are administered simultaneously, separately, or sequentially with one or more therapeutic agents. When used in such a combination the one or more therapeutic agents and the one or more flavonoid derivatives according to the present invention can be administered as separate agents at the same or different times or they can be formulated as a single composition comprising both compounds.

In some embodiments, a composition of methoxy flavonoids in linoleic acid containing SNEDDS may further comprise additional one or more cardiovascular agents, for example, other vasodilators such as aluminium cotinate, bamethan, bencyclane, bosentan, betahistine, bradykinin, brovincamine, bufeniode, buflomedil, calamine, cetiedil, ciclonicate, cinepazide, cinnarizine, cyclandelate, citicoline, cyclonicart, diisopropylamine chloroacetate, cofuranose, diltiazem, nicorandil, nitroglycerin, ebrunamomine, eledoisin, fenoxedil, flunarizine, hepronicate, ifenprodil, ibudilast, iloprost, inositol acinate, isoxsuprine, kallidin, kallikrein, moxisylyte, nicamate, nosergoline, nimodipine, nafronyl, nafuronyl nicametate, nicergoline, nofedrine, nylidrin, pentifylline, papaverine, phenoxedil, pentophilin, pentoxifylline, piribedil, prostaglandin E1 and I2, suloctidil, tolazoline, romeridine, xanthinol niacinate, vincamine, vinpocetine, vicidial and pentoxylphyllin.

As used herein, the term “poorly water-soluble” or “lipophilic” refers to having a solubility in water at 20° C. of less than 1%, e.g., 0.01% (w/v), i.e., a “sparingly soluble to very slightly soluble drug” as described in Remington, The Science and Practice of Pharmacy, 19^{th} Edition, A. R. Gennaro, Ed., Mack Publishing Company, Vol. 1, p. 195 (1995). Examples of therapeutic classes of therapeutic compounds include, but are not limited to, antihypertensives, antianxiety agents, anticlotting agents, anticonvulsants, blood glucose-lowering agents, decongestants, antihistamines, antitussives, antineoplastics, beta (β)-blockers, anti-inflammatories, antipsychotic agents, cognitive enhancers, anti-atherosclerotic agents, cholesterol reducing agents, anti-obesity agents, autoimmune disorder agents, anti-impotence agents, antibacterial and antifungal agents, hypnotic agents, antibiotics, anti-depressants, anti-Parkinsonism agents, anti-Alzheimer's disease agents, antiviral agents and combinations of the foregoing.

The flavonoid compounds disclosed herein are useful for a variety of purposes, including but not limited to: treatment and prevention of cardiovascular disease and regulated disease states, particularly, cholesterol or lipid related disorders, such as atherosclerosis, dyslipidemias, dyslipoproteinemias, hypertension, coronary artery disease, cerebrovascular disease, and the like. In addition, due to its structural resemblance to other polyphenols with known bioactive functions (e.g. resveratrol), flavonoids may be used for preventing and treating a disease associated with the presence of reactive oxidative species (ROS), such as ischemia, cancer, pulmonary vascular disease, diabetes, burns, chronic obstructive pulmonary disease, autoimmune disorders, septic and hypovolemic shock, hepatitis, emphysema and retinal vascular disease.

In preferred embodiments, the flavonoids are used as a treatment for acute hypertensive conditions such as stroke, myocardial infarction, mechanical trauma resulting from crush injury, vascular surgery, heart bypass or heart transplant surgery. The term “hypertension” refers to a medical condition that is characterized by an abnormal, elevated blood pressure and is diagnosed when a systolic blood pressure is above 140 mmHg and a diastolic blood pressure is above 90 mmHg. Systolic blood pressure is the maximum pressure in the artery and diastolic blood pressure is the minimum pressure in the artery between heart beats when the heart relaxes and fills with blood. An increase in systolic and/or diastolic blood pressure is closely monitored as a determinant of hypertensive event.

“Hypertension emergency” described herein is defined as a condition with significantly elevated systolic blood pressure that is greater than 180 mmHg or diastolic blood pressure greater than 120 mmHg with potential correlation with end-organ damage. The most affected organs include the brain, kidney, heart and lungs. Symptoms associated with hypertension emergency include headache, chest pain, focal neurologic deficits, altered mental status, SOB, pulmonary edema, renal failure, severe anxiety, agitation, abnormal sensation etc. In hypertensive emergency, the goal is to rapidly reduce the blood pressure to stop ongoing organ damage. Rapid elevations in blood pressure can trigger any of these symptoms. A plurality of tests for emergency hypertension diagnosis may be performed (e.g. urine toxicology, blood glucose measurement, a basic metabolic kidney evaluation, liver function evaluation, electrocardiogram (ECG), chest x-rays, pregnancy screening, retina examination, etc.), in addition to a basic blood pressure, heart rate, cardiac output, O₂ saturation, mean systemic arterial pressure and/or mean systemic venous pressure measurements.

In some embodiments, an increase in peripheral vascular resistance (systemic vascular resistance, SVR) or an elevation of vascular resistance afterload may be observed as a hypertensive symptom. SVR is the resistance in the circulatory system that is used to create blood pressure and the flow of blood. An increase in SVR may be due to vasoconstriction. In another embodiment, an increase in myocardial contractility and cardiac afterload may be hypertensive symptoms. Combinations of factors may lead to changes in contractility, such as increase in sympathetic stimulation, stroke volume, afterload, and heart rate. In yet another embodiment, shortened ventricular ejection time (LVET) as well as increased non-ejection time may be considered as hypertensive symptoms. Left ventricular ejection time (LVET) measure the period of blood flow across the aortic valve. LVET is also influenced by heart rate, preload, afterload, and contractile state. Reduced LVET may be caused by systolic or diastolic dysfunction, as LVET is proportional to stroke volume.

As used herein, the term “afterload” refers to a determinant of cardiac output and the pressure that heart must work against to eject blood during ventricular contraction. Afterload changes to adapt to the continually changing demands on the cardiovascular system and is proportional to mean systolic blood pressure.

In a preferred embodiment, the pharmaceutical composition of methoxy flavonoid and linoleic acid in a SNEDDS is administered to a subject in an amount sufficient to reduce the hypertensive symptoms described above without triggering reflex tachycardia.

An effective amount of a composition of the present invention sufficient to achieve a therapeutic or prophylactic effect should be determined by standard procedures used by medical professionals, e.g. physicians. The compositions described herein may be administered on multiple occasions. The interval between single doses can be daily, weekly, monthly, or yearly. Alternatively, the composition can be administered as a sustained release formulation. As noted above, dosage and frequency will vary depending on a plurality of considerations including the intended uses (i.e. prevention or treatment), efficacy and the half-life of the composition in a subject.

By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular, from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The active agent may be combined with pharmaceutically acceptable excipients. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The compounds described herein are generally delivered (administered) in a pharmaceutical composition and the present invention encompasses such formulations/compositions. The pharmaceutical compositions generally comprise at least one of the disclosed compounds, i.e. one or more than one (a plurality of) different compounds in a single formulation. The compositions also generally include a pharmacologically suitable (physiologically compatible) carrier, which may be aqueous or oil-based. In some aspects, such compositions are prepared as liquid solutions or suspensions, or as solid forms such as tablets, pills, powders and the like. Solid forms suitable for solution in, or suspension in, liquids prior to administration are also contemplated (e.g. lyophilized forms of the compounds), as are emulsified preparations. In some aspects, the liquid formulations are aqueous or oil-based suspensions or solutions. In some aspects, the compounds are mixed with excipients which are pharmaceutically acceptable and compatible with the compounds, e.g. pharmaceutically acceptable salts. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, and the like. If it is desired to administer an oral form of the composition, various thickeners, flavorings, diluents, emulsifiers, dispersing aids or binders and the like are added.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like.

Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

In a particular embodiment, the subject is an animal. The animal may be selected from the group consisting of humans, non-human primates, cattle, horses, pigs, sheep, goats, dogs, cats, birds, chickens or other poultry, ducks, geese, pheasants, turkeys, quails, guinea pigs, rabbits, hamsters, rats, and mice.

All percentages disclosed herein are in weight percent, unless otherwise indicated. It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided it is understood that each intervening value, to the tenth of the unit of the lower limit unless the context clearly dictates otherwise, between the upper and lower limit of that range and any other stated or intervening value in that state range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

### Example 1

### Methods and Materials

PEG 400, oleic acid and Tween® 80 were purchased from Sigma—Aldrich (St. Louis, Mo.). Linoleic acid was obtained purchased from Acros organics (Fair Lawn, N.J.). Angiotensin II and dimethyl sulfoxide (DMSO) from (Sigma-Aldrich, Munich, Germany) were used in the biological study.

### 1. Plant Material and Isolation of Active Compounds

Flowering aerial parts of Psiadia punctulata were collected from Al-Taif governorate, Kingdom of Saudi Arabia. The plant material was dried and extracted with methanol at room temperature. The collected methanol extract was evaporated, fractionated with chlorofrom, and ethyl acetate. Chloroform fraction was subjected to different column chroatography to get the major vasodilator methoxy flavonoids .

### 2. Development of IV Formulation and Preparation of Ternary Phase Mixtures

In this study, self-nanoemulsifying drug delivery system (SNEDDS) was utilized to develop the IV formulation. Polyethylene 400 was selected as a cosurfactant, tween 80 (polysorbate 80) was selected as a surfactant. Three oils namely, olive oil, linoleic acid and oleic acid were screened to select the oil that produces SNEDDS with the lowest globule size. Selection of the oils was based on their biocompatibility that render the formulation suitable for IV administration. Seven different formulations containing different quantities of oil, surfactant and cosurfactant were proposed and their composition is illustrated in table 1. Briefly, 1 gram of SNEDDS was prepared by accurately weighing the calculated amount of oil, surfactant and cosurfactant in an Eppendorf tube. Each mixture was vortex for 30 seconds until a homogenous dispersion was obtained. The total weight of the oil, surfactant and cosurfactant in any SNEDDS mixture was always added to 100%.

TABLE 1
Composition of the prepared self-nanoemulsifying drug delivery system
(SNEDDS) and the obtained results for the size and polydispersity index

| | | PEG | Tween | Size (nm) | | | PDI | | |
|---|---|---|---|---|---|---|---|---|---|
| | Oil | 400 | 80 | Olive oil | Linoleic acid | Oleic acid | Olive oil | Linoleic acid | Oleic acid |
| Run | % | % | % | SNEDDS | SNEDDS | SNEDDS | SNEDDS | SNEDDS | SNEDDS |
| 1 | 10 | 80 | 10 | 422 ± 15.62 | 233.33 ± 4.04 | 367 ± 14.73 | 0.897 ± 0.059 | 0.411 ± 0.032 | 0.889 ± 0.08 |
| 2 | 20 | 70 | 10 | 662.33 ± 59.80 | 446.33 ± 10.02 | 1095.33 ± 130.27 | 0.950 ± 0.036 | 0.909 ± 0.039 | 0.857 ± 0.13 |
| 3 | 30 | 60 | 10 | 1170.33 ± 142.46 | 793.67 ± 30.66 | 1288 ± 70.92 | 0.940 ± 0.043 | 0.507 ± 0.015 | 0.940 ± 0.04 |
| 4 | 40 | 50 | 10 | 1716.33 ± 225.75 | 865.33 ± 48.88 | 1690.67 ± 111.21 | 0.916 ± 0.081 | 0.972 ± 0.038 | 0.916 ± 0.08 |
| 5 | 50 | 40 | 10 | 5782.67 ± 431.37 | 1104 ± 59.19 | 2114.67 ± 203.85 | 0.561 ± 0.107 | 0.929 ± 0.097 | 0.995 ± 0.04 |
| 6 | 20 | 60 | 20 | 775 ± 81.05 | 333.33 ± 8.73 | 457 ± 39.94 | 0.994 ± 0.004 | 0.549 ± 0.094 | 0.834 ± 0.03 |
| 7 | 30 | 50 | 20 | 2064.33 ± 290.59 | 439.67 ± 22.47 | 631.33 ± 57.38 | 0.830 ± 0.146 | 0.820 ± 0.035 | 0.975 ± 0.03 |

### 3. Characterization of the Prepared SNEDDS

Known weight (1 g) of each SNEDDS formulation was added to 20 ml of distilled water on a magnetic stirrer until formation of a homogenous dispersion (nano-emulsion). The globule size and polydispersity index (PDI) of the obtained emulsions were determined using Malvern Zetasizer Nano ZSP, Malvern Panalytical Ltd (Malvern, United Kingdom). Dynamic light scattering with non-invasive backscatter optics was the technique used to measure the size. The average of three reading was recorded.

4. Preparation of the Medicated IV formulation

Known weight (2.1 mg) of the freeze-dried isolated compounds was separately added to 1 g of the selected SNEDDS formulation which contains 10% linoleic acid, 80% PEG 400 and 10% tween 80. The mixture was vortex until complete dissolving of the compound in the SNEDDS formulation. The medicated SNEDDS (1 g) was added to 20 ml double distilled water on a magnetic stirrer. Stirring was continue until formation of a homogenous mixture that was sterile filtered using Ministar® single use 0.45 mm, non-pyrogenic syringe filter of Sterile-ED, Sartorius Stedim Biotech GmbH (Goettingen, Germany) to prepare medicated IV formulations. Plain (non-medicated) IV formulation was also prepared for comparative study.

### 5. Biological Study

### 5.1 Animals

Six-week-old male Wistar rats with a weight of (250-275 g) were obtained from Zagazig University. They were kept in clear cages made of polypropylene and with good ventilation (3-4 rats in each cage), under constant environmental conditions of 22±2° C. temperature, 50-60% relative humidity and 12-h day and night cycle. Unlimited rodent pellet food and purified water were provided to the rats. The experimental design and animal handling procedures were as indicated by the guidelines of the Ethical Committee for Animal Handling at Zagazig University.

### 5.2 Blood Pressure Recording

The blood pressure was recorded invasively in real. The rats were subjected to anesthesia with single intraperitoneal injection of 100 mg/kg ketamine and 10 mg/kg xylazine. Animals' body temperature was held at 37° C. by means of a rectal probe and automated heating pads. A micro-tip pressure volume catheter (PV catheter, SPR-901, Millar Instruments, Houston, Tex., USA) was inserted via a small opening into the right carotid artery. This instrument can continuously monitor the arterial pressure. The micro-tip catheter was linked via a Power Lab Data Interface to a computer running the Lab Chart professional software (v8.0, AD Instruments, Bella Vista, Australia) incorporating blood pressure (BP) module. Following a stabilization time of 5 min, readings were continuously recorded. The BP module was employed to monitor the systolic and diastolic blood pressure.

### 5.3 Electrocardiogram (ECG) Recording

A Powerlab® system (AD Instruments, Bella Vista, Australia) linked to a computer running the LabChart professional software with the ECG module was employed to record the standard surface ECG according to the methodology outlined in a previous report by our group. The ECG module quantitatively assesses the various elements of the ECG.

### 5.4 Acute Induction of Hypertension

After 10 min (stabilization period) of basal recording of invasive blood pressure and ECG, angiotensin II (120 ng/min/kg) was slowly infused through the femoral vein using syringe pump and continued throughout the experiment duration.

### 5.5 Animals Treatment

After 10 min (stabilization period) of starting angiotensin infusion; the plain and the medicated IV formulations containing either compound 1 or 5 were injected into the femoral vein in doses of 12, 24, 36 μg/kg every 10 minutes in 0.3 ml injection volume. Invasive blood hemodynamics and ECG were continuously recorded throughout the experiment. Saline (0.3 ml) was injected in time control experiments.

### 5.6 Statistical Analysis

Values of the present study presented in form of mean ±SEM. Statistical analysis was carried out using the Prism 5 computer program (Graph Pad, USA). Statistical comparison was done using repeated measures Two way analysis of variance (ANOVA) followed by Bonferroni' post-hoc test of baseline-corrected data and P<0.05 was considered significant.

### Example 2

### 1. Characterization of Isolated Compounds

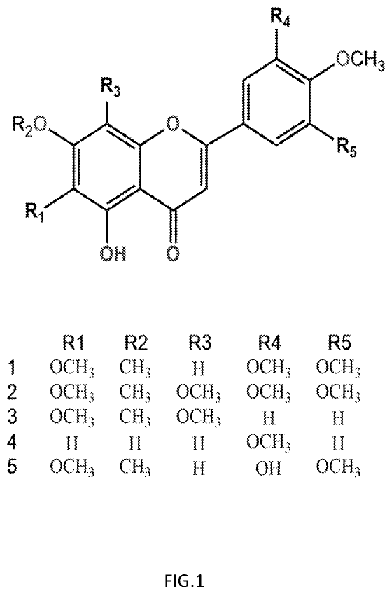

Isolated compounds were identified based on their NMR data and comparison with previously published data. In FIG. 1, the compounds were identified as umuhengerin (1), gardenin A (2), gardenin B (3), luteolin-3′,4′ -dimethyl ether (4), and 5,3′-dihydroxy-6,7,4′,5′-tetramethoxyflavone (5).

### 2. SNEDDS Formulation

Three different SNEDDS groups, of 7 runs each, containing PEG 400 as a cosurfactant, tween 80 as a surfactant and one of the studied oils (olive oil, linoleic acid, and oleic acid) were prepared and characterized for the globule size and PDI, and the obtained results are represented in table 1.

The aim was to develop a nano-emulsion with the lowest globule size. The united states pharmacopeia (USP) <729> stated that the mean globule size of an injectable emulsion should be less than 500 nm. The marketed intralipid 10% and 20% products have a mean globule size of 276 and 324 nm, respectively. In this study, as the concentration of oil was increased the globule size was increased, the effect that could be attributed to the availability of more surfactant molecules, at low oil concentration, that adsorb and form a closely pack surfactant film at the oil/water interface. This effect leads to formation of a stable system of low interfacial tension and hence small globule size. Based on the obtained results for particle size, SNEDDS formulation which contains 10% linoleic acid, 80% PEG 400 and 10% tween 80 was used to develop a medicated IV formulation containing the isolated compounds.

### 3. Biological Study

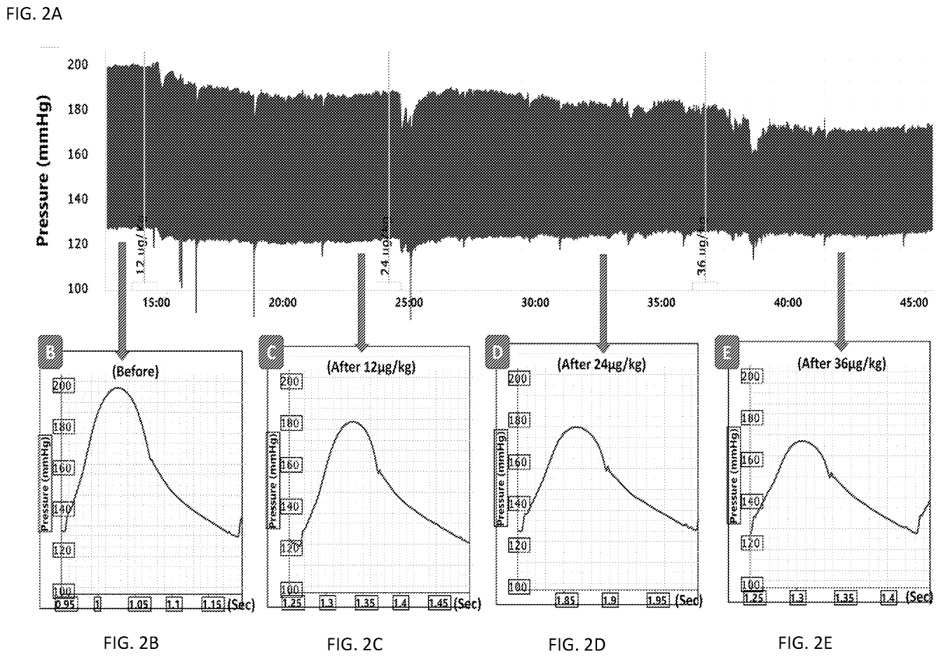

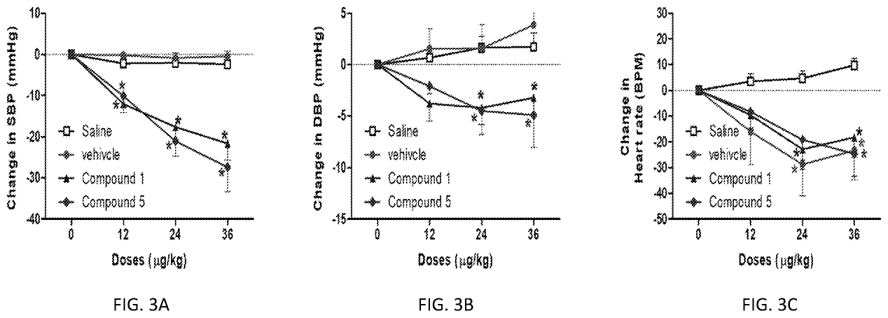

While compounds 1, 4 and 5 showed significant vasodilation activity in our previous report, the anti-hypertensive effect in the current study was observed only with compounds 1 and 5. Effects of formulations contain 1 or 5 on systolic and diastolic blood pressure and heart rate As shown in FIG. 2 and FIG. 3, intravenous injections containing either of formulas containing compound 1 or compound 5 in doses of 12, 24 and 36 μg/kg resulted in a gradual dose dependent reduction in the elevated systolic blood pressure induced by angiotensin after 10 min of each dose injection (p<0.05). The reduction in diastolic blood pressure was also gradual and reached a plateau with statistical significance at doses 24 and 36 μg/kg (p<0.05, FIG. 3B) compared to the group administered the plain IV formulation. Intravenous injection of the plain IV formulation resulted in significant reduction in heart rate compared to saline group, while the IV formulations containing compounds 1 or 5 did not produced any further effect on heart rate compared with the plain IV formulation. (FIG. 3C). The vasodilating effects of the P. punctulata isolated compounds 1 and 5 were observed, but this behavior does not guarantee the antihypertensive efficacy as evidence by the lack of antihypertensive efficacy of compound 4. Compounds usually have multiple effects and mechanisms, and the net result is a summation of all these effects. The carefully selected vehicle ingredients helped in having a vehicle with significant reduction of heart rate per se. Among many oils that can be used in SNEDDS preparation, linoleic acid with potential effect on heart rate was selected. This effect on heart rate rescues the vasodilating effect of compounds isolated from P. punctulata from being masked by the reflex tachycardia that commonly associated with the decrease in peripheral resistance by most vasodilators.

4. Effects of IV formulations contain 1 or 5 on pulse pressure, dicrotic notch pressure and SDP difference.

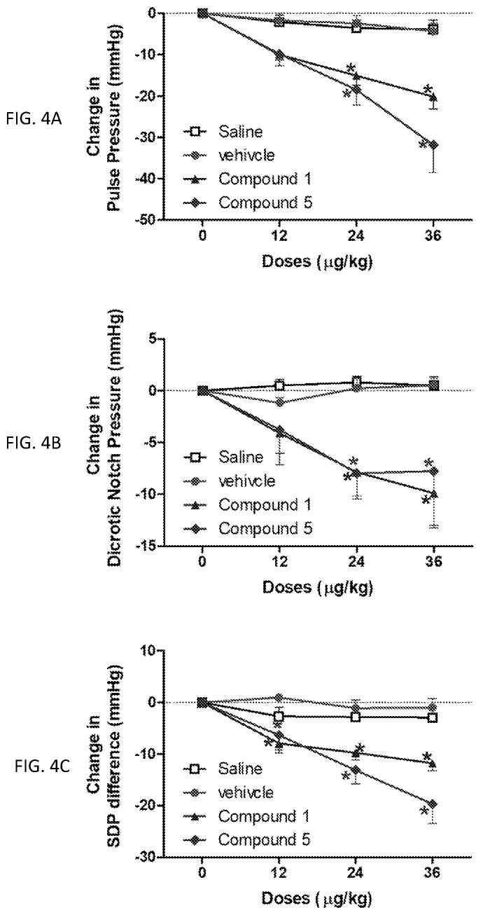

Both formulations contain compounds 1 and 5 resulted in significant reduction in both pulse pressure and dicrotic notch pressure started after 10 minutes of intravenous injection of doses 24 and 36 μg/kg compared to vehicle group (p<0.05, FIGS. 4A and 4B). Moreover, formulations contain compound 1 or 5 resulted in significant reduction in SDP difference compared to vehicle group at doses 12, 24 and 36 μg/kg (p<0.05, FIG. 4C). The dicrotic notch is a ubiquitous feature of the pressure waveform in the aorta. It is universally considered to be a marker of the end of aortic systole and the beginning of diastole and is used routinely for this purpose in clinical practice. Increased vasoconstriction is related to a higher dicrotic notch. Both formulations contain compound 1 or 5 resulted in significant reduction in dicrotic notch pressure compared to vehicle group. The appearance of the dicrotic notch was the single most important feature of the peripheral pulse wave and suggested a relationship between disappearance of the dicrotic notch and the presence of existing vascular disease.

The difference between systolic and dicrotic pressure (SDP difference), is important in reflecting the coupling between myocardial contractility and a given afterload. In the present study intravenous injection of formulations contain compound 1 or 5 resulted in significant reduction in SDP compared to vehicle injection.

5. Effects of IV formulations contain compound 1 or 5 on ejection duration, non-ejection duration and time to peak.

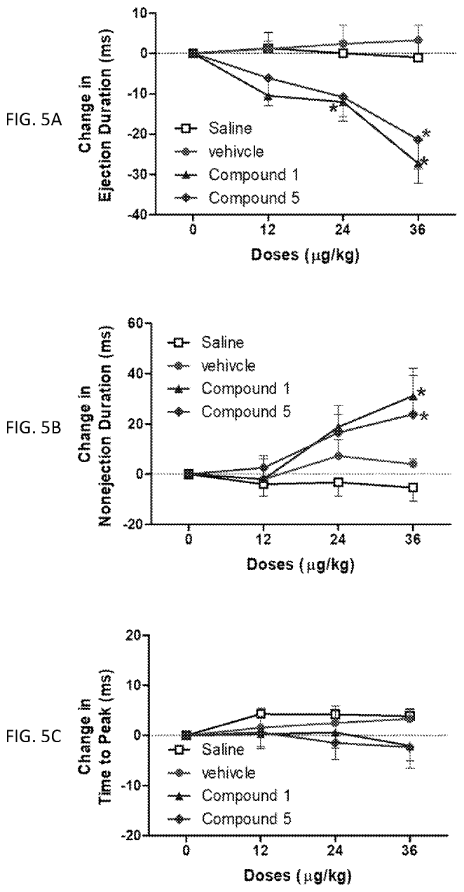

FIG. 5A-C show that Intravenous injection of formulation contains compound 1 resulted in significant decrease in ejection duration at doses 24 and 36 μg/kg and significant increase in non-ejection duration at dose 36 μg/kg but no change in time to peak compared to vehicle group. While formulation contains compound 5 resulted in significant decrease in ejection duration and significant increase in non-ejection duration at dose 36 μg/kg and didn't change in time to peak compared to vehicle group (p<0.05). Ejection duration reflects the time interval when blood is ejected into the aorta. However, the non-ejection duration reflects the periods of time when all heart valves are closed. Both formulations contain compound 1 or 5 intravenous injection decreased ejection duration and increased non-ejection duration without affecting time to peak in this model of hypertension. The ejection duration from the left ventricle has been significantly prolonged in the patients with central systolic BP higher than brachial systolic BP. Prolongation of ejection duration concurrently widens the pulse pressure, which leads to ventricular hypertrophy.

6. Effects of IV formulations contain compound 1 or 5 on cardiac electrophysiology.

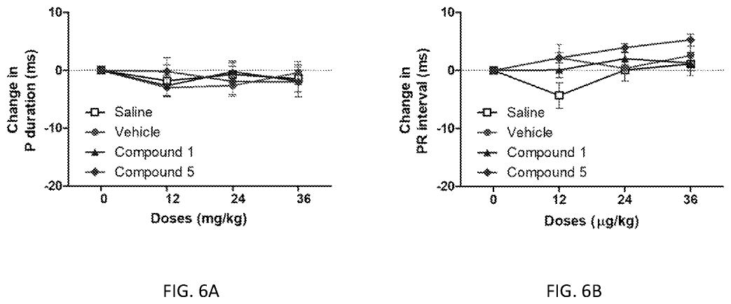

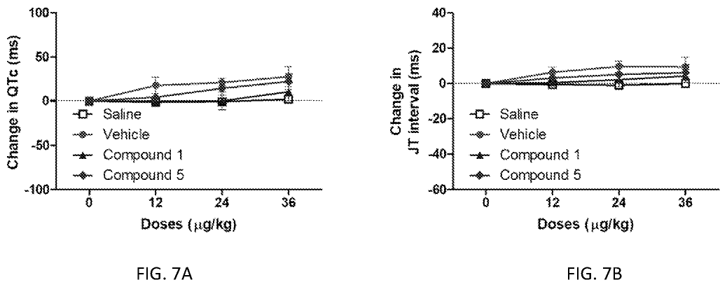

Injection of formulations contain compound 1 or 5 did not significantly affect atrial conductivity or the propagation of the impulse through the AV node and the conduction system to the ventricles as it did not affect P-wave duration and PR interval (FIG. 6). Similarly, ventricular activity from the beginning of ventricular depolarization through the plateau phase to the ventricular repolarization was not affected by intravenous injection of formulations contain compound 1 or 5 as it had no significant effect on either QTc or JT intervals (FIG. 7).

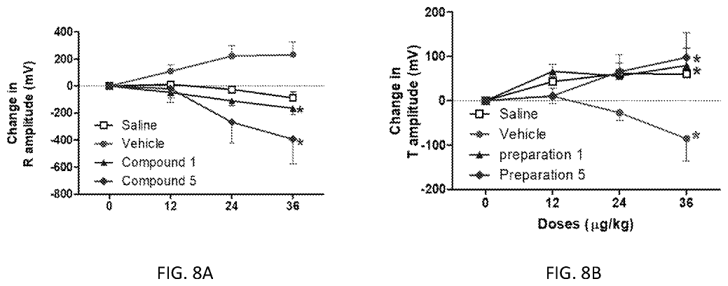

On the other hand, vehicle intravenous injection resulted in a reduction in T wave amplitude, this reduction is significantly different from saline group at dose 36 μg/kg (p<0.05, FIG. 8B). Both formulations contain compound 1 or 5 resulted in a significant decrease in R wave amplitude and increase in T wave amplitude compared to vehicle group only at dose 36 μg/kg of formulation 5 (p<0.05, FIG. 8)

Since hypertension is an asymptomatic and insidious disease, early ECG signs for cardiac electrical remodeling provide a wealth of information for disease stratification. Intravenous injection of formulations contain compounds 1 or 5 did not affect atrial conductivity, impulse propagation through the AV node and the conduction system to the ventricles or ventricular activity from the beginning of ventricular depolarization through the plateau phase to the ventricular repolarization in hypertensive rats infused with angiotensin II. However, their effect on cardiac conductivity were obvious on R wave amplitude and T wave amplitude which were significantly different from vehicle group at dose 36 μg/kg.

In conclusion, vasodilators produced reflex tachycardia and an increase in heart rate that masks its anti-hypertensive effect. Formulations containing compounds 1 or 5 in IV SNEDDS containing 10% linoleic acid caused significant antihypertensive effect through vasodilatation and decreasing peripheral resistance without reflex tachycardia. This could be detected through the ability of this formula to reduce the elevated diastolic, pulse pressure, dicrotic notch pressure and the systolic-dicrotic notch pressure difference. The studied nano-pharmaceutical formulation are thus effective medications in hypertensive emergencies, after clinical evaluation.

1-7. (canceled)

8. A method of treating hypertension while suppressing a reflex tachycardia in a subject in need thereof, comprising the step of administering to the subject a therapeutically effective amount of a self-nanoemulsifying drug delivery system (SNEDDS) composition comprising:
a) one or more methoxylated flavonoids having the structure of formula I or pharmaceutically acceptable salts thereof, 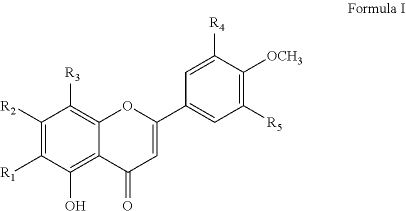
wherein R₁, R₂ and R₅ are OCH₃, R₃ is hydrogen, and R₄ is OH or OCH₃; and
b) linoleic acid at a concentration of 5-30 wt %,
wherein the one or more methoxylated flavonoids are at a dose of 1 μg/kg to 7 mg/kg.

9. The method according to claim 8, wherein the subject is a human or an animal suffering an acute hypertensive condition of a stroke, a myocardial infraction, a mechanical trauma resulting from a crush injury, a vascular surgery, a heart bypass surgery or a heart transplant surgery.

10. (canceled)

11. The method according to claim 8, wherein the symptom associated with hypertension further comprises an elevation of myocardial contractility and cardiac afterload.

12. The method according to claim 8, wherein the symptom associated with hypertension further comprises a reduced ventricular ejection time.

13. The method according to claim 8, wherein the symptom associated with hypertension further comprises an increased ventricular non-ejection time.

14. The method according to claim 8, wherein the hypertension is urgency or emergency hypertension.

15. (canceled)

16. The method according to claim 8, wherein the self-nanoemulsifying drug delivery system (SNEDDS) composition is administered intravenously.

17. The method according to claim 8, wherein the one or more methoxylated flavonoids are administered at a dose selected from 12-36 μg/kg.

18. The method according to claim 8, wherein the SNEDDS composition does not include olive oil.

19. The method according to claim 8, wherein the SNEDDS composition does not include oleic acid.
